(19) United States
(12) Patent Application Publication
(10) Pub. No.: US 2003/0219866 A1
(43) Pub. Date: Nov. 27, 2003

(54) Stem cell-like cells

(76) Inventor: Wiebe Kruijer, Leusden (NL)

Correspondence Address: TRASK BRITT, P.O. BOX 2550, SALT LAKE CITY, UT, 84110, US

(21) Appl. No.: 10/349,505

(22) Filed: Jan. 21, 2003

Related U.S. Application Data
(63) Continuation of application No. PCT/NL01/00561, filed on Jul. 20, 2001.

(30) Foreign Application Priority Data
Jul. 21, 2000 (EP) 00202634.2

(51) Int. Cl. C07K 14/475; C07H 21/04; C12P 21/02; C12N 5/08

(52) U.S. Cl. 435/69.1; 435/320.1; 435/366; 530/350; 536/23.5

(57) ABSTRACT

The invention relates to the field of embryology, embryogenesis, molecular genetics, (veterinary) medicine and zoo-technical sciences, and to the generation of stem cell-like cells. The invention provides a method for obtaining a stem cell-like cell from a sample taken from a multicellular organism, preferably an organism with some measure of differentiated tissue, thus preferably being beyond the morula stage, comprising culturing cells from the sample and allowing for transcription, translation or expression by at least one of the cells of a gene or gene product that in general is differentially expressed at the various different phases of embryonic development of the organism as described.

### CROSS-REFERENCE TO RELATED APPLICATION

This application is a continuation of PCT International Patent Application No. PCT/NL/01/00561, filed on Jul. 20, 2001, designating the United States of America, and published, in English, as PCT International Publication No. WO 02/08388 A2 on Jan. 31, 2002, the contents of the entirety of which is incorporated by this reference.

### TECHNICAL FIELD

The invention relates to the field of embryology, embryogenesis, molecular genetics, (veterinary) medicine and zoo-technical sciences, and to the generation of stem cell-like cells.

### BACKGROUND

A particular problem in embryology is the understanding of appearance, during development, of complexity of form and function where previously no such complexity existed. Historically two contrasting points of view have been held on this problem. One of these, the so-called theory of epigenesis, considered that during development there is actually the creation of new structures; whereas the other, the theory of preformation, maintained that a pre-existing diversity is already present in the fertilized egg (or in the sperm) and that future development consists merely in the unfolding and rendering visible of this innate diversity. The embryological investigation of the past hundred-and-fifty years have demonstrated most conclusively that the actual processes of development are of an epigenetic nature but the doctrine of preformation has been reintroduced, in a much modified form, in the explanation of the facts established by modern genetics.

Classically, the fundamental morphogenetic mechanisms during embryonic development are described under the headings of growth, differentiation and metabolism. Growth is increase in spatial dimensions and in weight; it may be multiplicative (increase in number of nuclei and (or) cells), auxetic or intussusceptive (increase in the size of cells) or accretionary (increase in the amount of non-living structural matter). Differentiation is seen as an increase in complexity and organization. This increase may be in the number of variety of cells and may not at first be apparent (“invisible” differentiation, e.g. determination of fates, segregation of potencies, but, when apparent (“visible” or “manifest” differentiation), constitutes histogenesis, the formation of differentiated or somatic tissue. Metabolism includes the chemical changes in the developing organism.

In the normal development of an embryo these fundamental ontogenetic processes are all closely interlinked, constituting an integrated system. They fit in with each other in such a way that the final product comes into being by means of a precise co-operation of reactions and events.

From a descriptive point of view the principal stages in embryological development leading to the formation of differentiated tissue within the developing organism can be described in stages. A first phase comprises maturation. The process associated with maturation of the female and male germ cells (gametes) including the reduction (meiotic) division. The mature gametes (ova in the female, spermatozoa in the male) are highly specialized cells which when fully differentiated do not usually live long unless they take part in fertilization.

Fertilization is the fusion of a female and a male gamete which results in the formation of the zygote or fertilized ovum. The zygote, although resulting from the fusion of two highly specialized cells, is typically regarded as being the most unspecialized (undifferentiated) of all metazoan cells, being the totipotent, pluripotent or stem cell from which a new individual and differentiated somatic (adult) tissue can develop. The process of differentiation, comprises forward differentiation from this toti- or pluripotent cell to specialized cells that have much more restricted potency but also transdifferentiation is observed where cells with distinct characteristics develop into cells with other distinct characteristics.

After fertilization the zygote soon undergoes repeated subdivision or cleavage by mitosis so that a number of cells, blastomeres, each much smaller than the ovum itself, is produced. After a certain number of cell divisions (generally at the 8-16 cell stage), the developing organism is called a morula. Typically, the individual cells of a morula, or at least the greater part of it, are still pluripotent, separation of cells from the morula stage can lead to the production of several new (cloned) individuals, stemming from one zygote, and at the morula stage the organism is in general seen as mainly comprising undifferentiated tissue. However, at some time at the end of cleavage the blastomeres are eventually grouped to form a hollow sphere of cells, the blastula or, in mammals, the blastocyst. In this blastula stage, the organism is in general seen as comprising for the first time some measure of differentiated tissue, whereby totipotent or pluripotent cells can mostly be found in the inner cell mass (TOM) of the blastula. Most of these cells also differentiate further, some of these become the so-called adult stem cells, but most differentiate into the specialized cell type to which they are being destined. A subgroup of stem cells, also called primordial germ cells, are kept in stock for the formation of gametes for a future generation. Experiments on, and intravitam staining of the blastulae of lower vertebrates, especially Amphibia, have made it possible to delimit the future fated of all regions of the blastula and thus to ascertain their potency, i.e., what localized areas become in normal development. The different areas of the blastula can thus be referred to as presumptive organ regions, e.g. one region is presumptive notochord, another presumptive neural plate, etc. It is the blastocyst, which in mammals proceeds to implantation in the uterus.

The blastula stage is succeeded by the gastrula which results from changes in position and displacements (morphogenetic movements) of the various presumptive regions of the blastula. In normal development the process of formation of the gastrula, or gastrulation, results in certain surface regions of the blastula becoming invaginated within the blastular cavity to form the endoderm, notochord and mesoderm, the tissue undergoes visible differentiation. The region through which invagination occurs is called the blastopore. The cells which remain on the blastular surface constitute the ectoderm, from which the epidermis and the neural plate are derived, and by their expansion and multiplication they gradually replace the areas of presumptive endoderm, notochord and mesoderm as the latter are invaginated. Gastrulation results in the establishment of the three primary germ layers, endoderm, mesoderm and ectoderm, again comprising, albeit somewhat mere differentiated, stem cells, and brings the presumptive organs of the embryo into the positions in which they will undergo their subsequent development. In reptiles, birds and mammals this gastrulation period is represented by the embryonic disc and primitive streak stages.

The gastrula stage is followed by the neurula stage in which the neural plate and the axial embryonic structures are elaborated. In Amphibia this is known as the neurula. This stage corresponds roughly to the somite stages in human development. At the end of the neurula, or somite stage of development the general pattern of the embryo is well established and later embryos are said to be in the so-called functional period of development.

The earlier embryonic stages, which have been described above, result in the appearance of the general embryonic pattern before the onset of specific function in the primordia of the different organs and tissues which are differentiated in these stages. Functions in the general sense are carried out at all times as all the cells are undergoing metabolic changes and must “work to live.” But with the onset of specific functions such as beating of the heart, contraction of muscles, secretion by glands, etc., the embryo enters on what may be called the functional period of development. Different organs, of course, commence to function at different times and no sharp distinction can be made between pre-functional and functional stages; growth and differentiation proceed in both. Nevertheless it is useful to consider the process of earlier stages as blocking out the main embryonic organ systems which will subsequently be elaborated under the influence of the specific functions which they perform. The functional influence does not, by any means, replace the genetically determined general pattern of development, but, in the development of many organs and tissues (e.g., the heart and blood vessels and the skeletal system), the effect of the function of an organ on its development is considerable. The functional stage of development results in the different organs and tissues coming into physiological relationship with each other and, therefore, in a degree of integration of total function which cannot exist in earlier stages.

The integration is facilitated, and indeed rendered possible, by the differentiation of the vascular and nervous systems and the onset of function in the endocrine glands. When the developing organism has entered such a functionally differentiated stage in its development, most if not all requirements underlying further developments in the following fetal, post-natal or adult stages of the individual have been met.

In short, all multicellular organisms are in general formed from a single pluripotent egg cell which give rise to further totipotent stem cells, the embryonic stem (ES) cells. ES cells are clonal cells, for example derivable from the inner cell mass of an developing blastula and are capable of adopting all the cell fates in a developing embryo. They form the pluripotent cells of the inner cell mass of mammalian pre-implantation embryos. These cells can in general be isolated and maintained in vitro as pluripotent cells or stem cell-like cells, and now can even give rise to new (cloned) individuals (see also http://www.nuffieldfoundation.org/bioethics).

Genetically modified mouse stem cells have been cultured together with feeder cells or co-cultured with mouse ES cells or cell aggregates such as embroid bodies to induce differentiation. However, to isolate or study the fate of stem cells, also under different inductive environments a cumbersome selection procedure is required. Such a selection method generally involves modification of stem cells by genetic means. Stem cells for example need essentially be modified to express a resistance marker (for example a neomycin resistance) gene in order to eventually eliminate the feeder or co-culture cells and select specifically for stem cells. For this technology to have application in medicine or even for zoo-technical applications, the use of a selection system based on the detection of a resistance marker is not desirable.

ES cells in general have an indefinite life span and can proliferate extensively when propagated under appropriate culture conditions involving the use of embryo-derived feeder cells or media containing lymphocyte inhibitory factor (LIF) (e.g., in the case of mouse ES cells). When, for example, injected into mouse blastocysts, in vitro propagated totipotent or pluripotent embryonic stem cells can contribute to all tissues of the recipient embryo as well as to the germ line and be a source of new (cloned) individuals. In addition, these pluripotent embryonic stem cells can be induced to differentiate in vitro yielding differentiated derivatives representative of all three germ layers including neuronal, myocardial and endothelial cells.

Functionally differentiated organisms with differentiated tissues such as mesodermal, ectodermal, endodermal, or even adult or somatic tissues may still contain a variety of cells that have normal functions in tissue such as the continuous generation of new cells, also in response to injury and aging (i.e., cell renewal). Such “differentiated or adult stem cells” have for example been found in bone marrow, bone marrow stroma, muscle, and brain. Adult somatic stem cells have similar, albeit some restricted capacity of self-renewal and may give rise to daughter cells with the same potential as well as daughter cells with a more restricted differentiation capacity. The differentiation potential of stem cells in differentiated tissues is in general thought to be limited to cell lineages present in the organ from which they were derived, excluding of course the potential of those primordial germ cells that are required for gamete formation.

### BRIEF SUMMARY OF THE INVENTION

This concept is rapidly changing as somatic stem cells, shown herein, are in fact highly plastic cells amenable to change given the appropriate environment, not acting only in tissue in which they reside, but may be recruited out of circulation and enter in regenerating of tissues at distal sites.

The invention provides a method for obtaining a dedifferentiated or transdifferentiated stem cell-like cell from a sample taken from a multicellular organism, preferably an organism with some measure of differentiated tissue, thus preferably being beyond the morula stage, comprising culturing cells from the sample and allowing for transcription, translation or expression by at least one of the cells of a gene or gene product that in general is differentially expressed at the various different phases of embryonic development of the organism as described above. Measuring Oct4 expression is a bona fide marker for determining the presence of dedifferentiated pluripotent human ES-equivalent cells. The same is true for expression of the markers such as SSEA1, SSEA3, TRA-1-60, Tra-1-81 or alkaline phosphatase. Dedifferentiated ES-equivalent cells are different with respect to isolated hES cells in that ES-equivalent cells are essentially feeder cell independent for proliferation as ES-equivalent cells. Dedifferentiated ES-equivalent cells have similar therapeutic potential. Hereby, the invention provides for a dedifferentiation and/or selection of at least one or some cells from the sample for stem cell-like cell characteristics based on for example the transcription (and possible further translation) of distinct gene products or the presence of distinct transcription factors (detectable by for example detecting relevant promoter activity or detecting other relevant gene products such as mRNA or (poly)peptides derived from a gene that is for example differentially expressed at the morula stage versus the neural stage, or the blastula stage versus the functional stage, or the blastula stage versus the adult stage. By allowing the cells from an already forward differentiated or specialized tissue to again or preferentially transcribe early phase genes that are no longer transcribed, or alternatively, to suppress or downregulate transcription of later phase genes, the dedifferentiation or selection of a stem cell-like cell is provided, even from an already functionally differentiated organism as described above. The invention thus provides a dedifferentiated stem cell or stem cell-like cell, and cell cultures or cell lineages derived thereof. The pluripotency of stem cell-like hES-eq cells has been established by production of teratomas following transplantation in immunodeficient (SCID) mice, and by injection of hES-eq cells in mouse blastocyst and assessment of the level of chimerism based on (i) detection of human or isogenic cell surface marker; (ii) the expression of human genes in developing tissues using RT-PCR and human gene-specific primers and (iii) expression of a hES-eq expressed reporter (GFP, LUC, LacZ) following stable or transient expression of this gene in hES-eq cells, followed by co-culture with undifferentiated cells such as inner cell mass derived cells, for example with aggregates of such cells or injection of hES-eq cells into the amniotic cavity of chick stage 4 embryo's and detection of chimerism in the developing tissues by analysis of cell surface marker expression and RT-PCR using human gene-specific primers.

Totipotent dedifferentiated stem cells as provided herein are obtained from for example human tissue. These hES-eq(uivalent) cells are characterized by expression of the stem cell-specific transcription factors Oct4, Sox2 and UTF1, specific pattern of expression of the cell surface markers stage-specific embryonic antigens SSEA-1 and SSEA-3, TRA-1-60 and TRA-1-81 and alkaline phosphatase, specific gene expression profiles as determined by DNA gene expression micro-arrays, and the capacity to form differentiated derivatives from embroid bodies following aggregation in the presence of retinoic acid or DMSO and expression of cell lineage markers (depending of the treatment) Troma-1 (endoderm derivatives), Neurofilament type-1 (neuronal derivatives), Cardiac Myosin Heavy chain (cardiac muscle), expression of telomerase activity and normal karyotype corresponding with the sex of the donor. From other mammals, similar ES-cells were obtained demonstrating similar (species-specific) characteristics.

Other procedures provided herein and allowing facilitating the recovery of dedifferentiated stem cells or stem cell-like cells involve manipulation of gene expression affecting cell cycle progression in the GO-GI phase of the cycle including pre-treatment with transforming growth factors for transient induction of cell cycle arrest, or timed addition of extracellular factors that block differentiation at early stages of development i.e., nodal to antagonize BMP effects or contribute to dedifferentiation, such as by Trichostatin A (TSA), and by removal of differentiating agents such as retinoids from Fetal Calf Serum (FCS) containing media, or timed addition of extracellular factors that sustain the proliferation of ES-like cells, i.e. LIF, growth factors including FGF's, PDGF's and interleukins, or co-culture of selectable adult stem cells as indicated above with pluripotent ES cells (mammalian, human, primate) classified as such; human embryonal carcinoma cells classified as such, i.e., N-tera-2; yolk-sac tumor cell lines classified as such, or culture of somatic stem cells with conditioned medium derived from the above cells lines, or isolated factor(s) present in the conditioned medium of the cell lines indicated, above, in particularly in those overexpressing UTF1. In the case of the production of dedifferentiated stem cells from humans, tissue samples were obtained under condition of (patient) informed consent and experiments involved controlled laboratory processes. The isolation of human ES equivalent (hES-eq) cells from pre- and post-natal and adult human tissue may further involve the following isolation and selection of hES-eq cells on the basis of expression of Oct4 promoter driven cell surface marker(s) (Schoorlemmer et al., Mol. Cell. Biol. 14:1122-1136, 1994) allowing specific recognition of the cell surface expressed molecules by specific antibodies. Isolation procedures may involve separation of hES-eq cells with magnetic bead or fluorescence activated cell sorting (FACS). Oct4-promoter driven expression of Green Fluorescent Protein (GFP) and isolation of GFP-expressing cell by FACS is provided as well Oct4-promoter driven expression of the neomycin resistance gene and selection of G418 resistant cells is optional.

Such a dedifferentiated stem cell, or stem cell-like cell, as provided herein can be used for all purposes that seem fit as use for stem cells in general, but offer also distinct advantages beyond current available stem cells. In one specific embodiment, it is now provided to take a sample from an individual suffering from a disease (in particular helpful, when the organism is human, in human medicine for, for example, bone-marrow deficient patients, patients suffering from Parkinson disease or Alzheimer's disease or diabetes or other disease where suppletion or transplantation with specialized cells is contemplated) or otherwise in need of transplant treatment, treat the sample as described herein, obtain a dedifferentiated stem cell from the individual, grow it into a culture of stem cell-like cells, provide, if required, for forward differentiation of the culture towards a more differentiated or required specialized cell type, and use such a culture or parts thereof as graft for treatment of the exact same individual. No major adverse immune response are to be expected when the graft is, so to speak, put back into the individual, however now provided with desired functionality's deemed necessary for treatment. In particular, the cells need no recombinant engineering to provide an immunological match with the recipient, the recipient being also the donor of the cells to begin with. In other words, the recipient can be his or her own donor.

A cell as provided herein can also be used to grow distinct tissue types, such as (heart) muscle cells, blood cells, blood, vessel cells, cartilage or bone tissue, neural cells, skeletal tissue, and so on, for which, again no immunological match is required when placed back into the donor/provider of the source of the graft. Of course, these cells lend themselves also to the provision of the immunological matches in case other recipients are contemplated, using methods known in the art and classical employed with the embryonic stem cells that give rise to specialized tissue or cells.

Of course, a cell as provided herein finds its use also in providing new (cloned) individuals, which is in particular advantageous, when the organism is a vertebrate such as a fish (salmon, trout, eel), poultry (chicken) or mammalian (mice, rats, guinea pigs, or other small laboratory animals, or farm animals like ruminants or pigs) in the field of the creation of (near identical or cloned) experimental animals or farm animals or animals for the production of xenotransplant-tissue (often pigs are used) or other desired (recombinant) products. Again, a major advantage of the method as provided herein lays in the fact that such cells can now be obtained from functionally differentiated or even specialized adult somatic tissue, such as muscle, brain, blood, none marrow, liver, mammary gland, and so on, allowing to first select the desired animal from amongst other related but less desirable animals (e.g. on production characteristics), and than cloning it, using for example an easily obtainable tissue biopsy as sample for the provision of the desired stem cell-like cell or cells from which cloning can commence. Such dedifferentiated cells as provided herein can, if required in an intermediate step, be injected into a (if desirable an unrelated) developing embryo (preferably blastula stage) and develop into a chimeric organism from which primordial germ cells or gametes with the desired specificity can be harvested, but can also be used for direct embryonic development.

In a preferred embodiment, a method according to the invention is provided wherein the somatic cells from the sample are cultured in the relative absence of a differentiation factor such as different members of the steroid-hormone receptor superfamily (nuclear receptors). ARP-1, RAR (retinoic acid receptor) but preferably in the relative absence of retinoid or retinoic acid or analogue thereof. This allows for routing the cell back to a dedifferentiated or totipotent state, as characterized by the differential expression of differentiation factor or retinoic acid induced or suppressed genes or fragments thereof. Culture medium can be deprived of retinoids or retinoid activity by for example charcoal filtration of the medium itself or its constituting components such as the serum (preferably bovine calf serum, preferably free of specified pathogens is used), measuring resulting activity and using the medium sufficiently deprived of the activity. Growth media, such as synthetic media, are otherwise produced and used as known in the art of cell culture, in particular of stem cell culture.

The invention also provides further comprising a selection method preferably not based on the genetic modification of somatic cells for the identification of embryonic stem cell-like cells from amongst a population of cells in adult somatic tissue. Dedifferentiated adult somatic cells which have this broad differentiation repertoire are herein also referred to as a somatic stem cell embryonic stem cell equivalent (SSCES-eq or stem cell-like cell. SSCES-eq have characteristics that are very similar or even indistinguishable from embryo-derived cells (ES) and have a developmental repertoire that is close or even identical to that of ES cells. This system is based on the observation that specific marker genes such as the well known transcription factor Oct 4 and for example its targets kFGF, UTF1 and SMAD regulated target genes are differentially expressed during the developmental processes observed in the growing embryo.

These dedifferentiated SSCES-eq can be multiplied in vitro and can under the right circumstances give rise to an almost unlimited source of stem cells to be used in a variety of ways. Dedifferentiated somatic stem cells from a single donor can be made recipient-independent and broad range applicable by genetic inactivation in vitro of the MHC locus. Therefore this invention provides the means to treat more easily individual patients, in a strict donor-recipient relationship, with SSCES-eq cells derived from their own tissues with properties equivalent to ES cells specialized for a given task. Furthermore it provides the means to treat various diseases in different affected individuals with general source of dedifferentiated SSCES-eq cells. The invention shows that adult somatic stem cells, although more specialized than pluripotent ES-cells can be used as alternative source for embryo-derived ES-cells, for the purpose of repairing or replacing body tissues (for example blood, nerve and myocardial tissues), with the main advantage that immunological matching is not required.

The present invention provides a method for in vitro selecting a somatic stem cell-like cell from differentiated tissue material or samples comprising culturing cells from the material under conditions allowing for induction of expression of essential pre-implantation (early blastocyte stage in mammals) gene products anchor suppression of expression of non-essential pre-implantation gene products. A somatic stem cell or tissue herein refers to any differentiated “body” cell or tissue be it of mesodermal, endodermal or ectodermal descent (for example blood, immune system, nerve, myocardial, muscle, intestinal tissue). Further the invention provides a method for in vitro selecting a somatic stem cell-like cell from post-implantation material comprising culturing cells from the material under conditions allowing for induction of expression of essential post-implantation gene products and/or suppression of expression of non-essential pre-implantation gene products.

The invention provides a method of selection of a stem cell-like cell (SSCES-eq) based on detecting differences in gene expression patterns between genes differentially expressed at different stages of embryonic development, in mammals for example identifiable as pre- and post-implantation stages. Methods to detect differential gene expression patterns are known in the art and comprises methods aimed at detecting “nucleic acid” and/or “amino acid.” “Nucleic acid” herein refers to an oligonucleotide, nucleotide or polynucleotide, and fragments or portions thereof, and to DNA or RNA of genomic or synthetic origin which may be single- or double-stranded, and represents the sense or antisense strand. “Amino acid” herein refers also to peptide or protein sequence. Included, in the scope of the invention is detection of different alleles of the polypeptide encoded by nucleic acid sequences or gene of interest. As used herein, an “allele” or “allelic sequence” is an alternative form of a polypeptide. Alleles result from a mutation (e.g., a change in the nucleic acid sequence, and generally produce altered mRNA or polypeptide whose structure or function may or may not be altered). Any given polypeptide may have none, or more allelic forms. Common allelic changes that give rise to alleles are generally ascribed to natural deletions, additions or substitutions of amino acids. Each of these types of changes may occur alone, or in combination with the others, one or more times in a given sequence. Deliberate amino acid substitution may be made on the basis of similarity in polarity, charge, solubility, hydrophobicity, and/or the amphipathetic nature of the residues as long as the biological activity of the polypeptide is retained. A “deletion” is defined as a change in either nucleotide or amino acid sequence in which one or more nucleotides or amino acid residues, respectively, are absent. An “insertion” or “addition” is that change in nucleotide or amino acid sequence which has resulted in the addition of one or more nucleotides or amino acid residues, respectively, as compared to the naturally occurring polypeptide(s). A “substitution” results from the replacement of one or more nucleotides or amino acids by different nucleotides or amino acids, respectively. Included is a polypeptide variant. A “variant” of a poly-peptide is defined as an amino acid sequence that is different by one or more amino acid “substitutions.” A variant may have “conservative” changes, wherein a substituted amino acid has similar structural or chemical properties, for example, replacing leucine with isoleucine. More rarely, a variant may have “non-conservative” changes (e.g., replacement of a glycine with a tryptophan). Similar minor variations may also include amino acid deletions or insertions, or both.

Methods to detect differential gene expression patterns are known to those skilled in the art. These procedures include, but are not limited to DNA-DNA or DNA-RNA hybridization. The form of such quantitative methods may include, Southern or Northern analysis, dot/slot blot or other membrane based technologies; PCR technologies such as DNA Chip, Taqman®, NASBA, SDA, TMA, in situ-hybridization, protein bioassay or immunoassay techniques ELISA, IFA and proteomic technologies. Other evolving technologies such as “metabolomics” can be employed to look at changes in metabolic profiles between tissues and/or cell types.

The invention also provides a method of selection whereby the material is derived from vertebrate tissue and/or cells. The term “vertebrate” refers to a life form having a spinal column. One preferred embodiment is that the somatic stem cell-like cell (SSCES-eq) selected is from adult somatic tissue. Included in the scope of the invention are somatic stem cells obtainable from primate somatic tissues. “Tissue” herein refers to a collection or aggregate of individual cell types. The invention further provides a method of selection whereby the tissue comprises muscle tissue and/or bone marrow tissue and/or bone marrow stroma and/or nerve tissue and/or brain tissue and/or blood. Unlike ES cells the dedifferentiated stem cells as provided herein can be derived from a multitude of tissue types. These cells are also derivable from bone marrow, bone marrow stroma, muscle and brain tissue. The invention further provides a method of selection whereby the tissue is adult tissue. One preferred embodiment is that the somatic stem cell-like cell is obtainable from nerve tissue and/or bone marrow tissue and/or bone marrow stroma and/or muscle tissue and/or brain tissue and/or blood, more preferably human. The invention further provides a method of selection comprising selecting the cell from the ectoderm and/or mesoderm and/or endoderm layer of the tissue. The invention further provides a method of selection comprising selecting the cell by detection of expression of cell-specific transcription factors Oct4 and/or Sox2 and/or UTF1 or homologues or orthologues thereof. The tissue-specific transcription factors Oct4 and/or Sox2 and/or UTF1 are required for the development of a precise somatic stem cell lineage. Hence the dedifferentiated stem cell provided herein can be identified on the basis of expression of these stem cell-specific transcription factors. The definition “homologue” is a term for a functional equivalent. It means that a particular subject sequence varies from the reference sequence by one or more substitutions, deletions, or additions resulting in ‘amino acid’ that encode the same or are functionally equivalent, the net effect of which does not result in an adverse functional dissimilarity between the reference and the subject sequence. Orthologues are similar genes found with other species.

The invention further provides a method further comprising selecting the cell by detection of expression of cell surface markers stage specific embryonic antigens SSEA-1 and/or SSEA-3 and/or TRA-1-60 and/or TRA-1.81 and/or alkaline phosphatase or analogs thereof. The invention further provides a method of selection whereby the somatic stem cell-like cell has telomerase activity. The invention further provides an isolated somatic stem cell obtainable from adult somatic tissue having telomerase activity. “Telomerase” activity herein refers to the activity of a specific enzyme termed telomere terminal transferase which is involved in the formation of telomere DNA. Telomers are required for replication and stable inheritance of chromosomes. The invention further provides a method of selection whereby the somatic stem cell-like cell has transdifferentiation capacity. Adult somatic stem cells have a high transdifferentiation capacity. By transdifferentiation capacity it is meant that somatic stein cells have the capacity of indefinite self-renewal by producing a multitude of daughter cells through repeated divisions. They give rise daughter cells with the same potential, as well as daughter cells with a more restricted differentiation capacity. Neuronal stem cells can give rise to blood cells after transplantation into the blood of irradiated stains of mice. Also muscle progenitor cells have been shown to be capable of transdifferentiation into blood. Furthermore, bone marrow stroma cells transplanted to the brain can generate astrocytes and hematopoietic stem cells can give rise to myocytes. The invention further provides a somatic stem cell-like cell comprising recombinant nucleic acid. Classically, ES cells are seen as extremely useful for creating transgenic animals, the dedifferentiated stem cell as provided herein is equally suitable. Methods to transduce stem cells are known in the art. A “gene delivery vehicle” herein is used as a term for a recombinant virus particle or the nucleic acid within such a particle, or the vector itself, wherein the vector comprises the nucleic acid to be delivered to the target cell and is further provided with a means to enter the cell.

The invention provides an isolated stem cell-like cell obtainable through selection capable of clinical use. A dedifferentiated stem cell or cells from a single donor can be made recipient-independent and broad range applicable by genetic inactivation in vitro of the MHC locus. Included in the scope of the invention is a pharmaceutical composition comprising a somatic stem cell-like cell or culture transduced with a gene delivery vehicle, to generate different tissue types for application in gene therapy. One usage is the generation of different types of tissue and/or tissue renewal. Another is the repair and/or replacement of different types of tissue. These tissues derived from somatic stem cell-like cells could be administered to a patient by transplantation into host tissue or by grafting for use in the treatment of by way of example Parkinson disease and/or cardiovascular disease and/or liver disease. For example adult somatic stem cells or the dedifferentiated pluripotent ES-like cells can be obtained for muscle tissue or cordial blood of a given patient donor, dedifferentiated (multiplied) in vitro, and can then be applied for brain tissue repair as donor recipient. This source of stem cells can be used in any kind of tissue renewal and/or repair and/or replacement in cases such as, but not limited to Parkinson disease, cardiovascular diseases, liver disease. Another preferred embodiment of the invention relates to the production of non donor-specific pluripotent ES-like cells for use in non-donor recipient tissue repair and/or renewal and/or replacement treatments. Dedifferentiated somatic stem cells from a single donor can be made recipient-independent and broad range applicable by genetic inactivation in vitro of the MHC locus. This has an advantage in that a general source of human stem cells can be applied for tissue repair anchor renewal and/or replacement treatments without tissue rejection problems arising. The invention further provides use of a cell or culture or graft or animal as provided herein in studying the biology of vertebrate development, in transplantation, in drug screening and drug discovery and in cosmetic surgery, whereby again immunological mismatches can be avoided. Included in the scope of the invention are cross species recipients.

Oct4, a member of the Pou domain, class 5, transcription factors (Pou 5fl) (Genbank accesion S68053) is one of the mammalian POU transcription factors expressed by early embryo cells and germ cells. It is a marker for PGCs and pluripotent stem cells in mammals. The activity of OCT4 is essential for the identity of the totipotent founder cell population in the mammalian embryo. Oct4 determines paracrine growth factor signaling from stem cells to the trophectoder, involving the Oct4/Sox2 target gene FGF4. Oct4 is a transcription factor whose expression is associated with an undifferentiated cell phenotype in an early mouse embryo and is downregulated when such cells differentiate. Expression of Oct4 in embryonic stem cells is controlled by a distal upstream stem cell specific enhancer that is deactivated during retinoid or retinoic acid (IRA) induced differentiation by an indirect mechanism in general not involving binding of RA receptors. The enhancer is thought to contain no retinoic acid receptor (RA) binding sites. Oct4 is subject to negative regulation by other differentiation factors such as different members of the steroid-hormone receptor superfamily (nuclear receptors). ARP-1, RAR (retinoic acid receptor). It has been shown that negative regulation of OCT4 expression during RA induced differentiation of embryonic stem cells is controlled by two different mechanisms, including deactivation of the stem cell specific enhancer and by promoter silencing by orphan hormone receptors.

Oct4 in combination with its co-regulator Sox2 binds to juxtaposed Oct4-Sox2 DNA binding sequences in promoters of a variety of target genes including FGF-4, PDGF-alpha, Rex-1 and UTF1. UTF1 binds to the SMAD binding element (SBE) consisting of the sequence CAGACAG or variants or thereof, which are present in TGF-beta/activin/BMP target genes to mediate SMAD-dependent transactivation. The 3 central nucleotides GAC of the SBE sequence are essential for both Smad as well as UTF1 binding. UTF1 forms complexes with the receptor-regulated Smads 1, 2, 3 and co-Smad4. Furthermore, overexpression of UTF1 blocks Smad-dependent transcriptional activation of TGF-beta, Activin and BMP target genes which include the developmental control genes Mix-1 and goosecoid expressed during gastrulation, the cell cycle inhibitors p15, p16 and p21 which block cell cycle progression, genes promoting cell adhesion like collagen, inhibitors of adhesion protein degradation including plasminogen activator inhibitor and the immediate early Fos/Jun genes which play a role in cell proliferation. A variety of Smad target genes are repressed through histon de-acetylase activity (EIDAC) as demonstrated by activation following treatment with Trichostation A.

Like Oct4, the expression of UTF1 is confined to embryonic stem cells. The relationship between expression of Oct4 and its direct target UTF1 implies that loss of expression of Oct4, activates TGF-beta/Activin/BMP signal transduction and target gene regulation. Similarly, over-expression of UTF1 blocks Smad-dependent target gene regulation and ES/EC cells differentiation. Alternatively, over-expression of UTF1 in adult stem cells may resulting in dedifferentiation resulting in cells with higher Oct4 expression resembling ES-equivalent cells.

Leukocyte inhibitory Factor (LIF) is a cytokine that acts through the JAK/STAT3 signal transduction pathway. Components involved in LIF signal transduction include the transmembrane LIF receptor and its dimerizing partner gp130, the tyrosine protein kinase Jak-2 and transcription factor STAT3. LIF also activates the ERK or JNK/p38 pathways downstream of gp130 receptors. IL-6 signal transduction involves the IL-6 receptor gp80 and further the components involved in LIF signal transduction. LIF signal transduction supports self-renewal and feeder-independence of mouse ES cells. This in contrast to human (primate) ES cells that are resistant to the action of LIF and fail to activate STAT3 in these cells. This differential sensitivity to LIF may be attributable to high expression of SOCS1, a negative regulator of LIF signal transduction acting at the level of JAK tyrosine kinase activation. The components of LIF signal transduction are expressed at similar levels in human ES and human mesenchymal stem cells. gp80 expression however is expressed in hMSC but not in human ES cells. Dedifferentiation of hMSC to ES-equivalent cells therefor results in loss of expression of this gene.

In mouse ES cells, LIF signal transduction induces, either directly or indirectly, the expression of Sox2, thereby modulating Oct4-Sox2 transcriptional activation, including the expression of UTF1. Regulation of Sox-2 expression in the human ES cells is presently unknown.

Oct4, Sox2 and UTF1 are components of a regulatory system that controls pluripotency of embryonic stem cells. Retinoids downregulate Oct4 which induces differentiation. LIF deprivation also induces differentiation affecting the expression of Sox2 at least in the murine system. Oct4 and/or Sox2 down-regulation UTF1 which allows Smad-dependent transcriptional activation affecting a variety of cell functions characteristic of the differentiated state. In adult stem cells, these effects are reversible leading to dedifferentiation and ES-equivalent cells characterized by normal OCT4 expression levels.

In Niwa Hitoshi et al., Nature Genetics 4, 372-376 (2000), the role of OCT4 as gatekeeper of embryonic stem cell pluripotency and its role in forward differentiation of ES has been investigated. However, the present invention provides the insight that regained Oct4 expression is linked to dedifferentiation back to embryonic stem cell pluripotency, in particular increasing Oct4 expression allows dedifferentiation of a more mature adult cell into the desired stem cell, the fact being that Oct4 expression is now defined as a useful marker for analysis of dedifferentiation of adult (stem) cells to ES-equivalent or ES-cell-like cells. Loss of Oct4 expression results in loss of pluripotency of embryonic stem cells and differentiation into trophoectoderm. Saijo Yukio et al., Genes to Cells, 1, 239-252 (1996), describe the isolation a number of pluripotent cell-specific downstream target genes of Oct4 that are differentially expressed between undifferentiated pluripotent cells and adult (stem) cells, and that are also useful markers for the purposes of a method as provided by the invention. Stewart, C. L., Nature Genetics 4, 328-330 (2000), also highlights the role as Oct4 as gatekeeper of pluripotency and control of embryonic stem cell development and differentiation. However, Stewart does not link overexpression of Oct4 with dedifferentiation of an adult or somatic stem cell to an ES-equivalent or ES-like cell. In Nishimoto Masazumi et al., Mol Cell Biol. 19, 5453-5465 (1999) the UTF1 gene was identified as a target for the transcription factor Oct4 acting in concert with the transcription factor Sox2. UTF1 is expressed in pluripotent stem cells and also possibly involved in maintenance of the pluripotent state. Consequently, also UTF1 is a useful marker for pluripotency of dedifferentiation as provided herein, while over-expression in adult cells may lead to dedifferentiation to ES-equivalent or like cells. PCT International Publication WO 00 27995 describes the isolation and characterization of human Embryonic Stem cells, which however are not obtained by dedifferentiation. OCT4 and SSEA4 have been used as markers to define the pluripotency of the isolated cell lines. The isolated human ES cell requires feeder cells for proliferation as undifferentiated ES cells.

### BRIEF DESCRIPTION OF THE DRAWINGS

FIG. 1: Identification of a RT-PCR primer set that specifically amplifies OCT4 in mixed mouse/human RNA samples. M: Mw markers; P19 EC: mouse EC cell line; P19 EC UTF: UTF1 expression in P19 mouse EC cells; NteraD2: human EC cell line; hu ES: human Embryonic Stem cells; huMSC: human Mysenchymal Stem Cells; beta2 micro-globulin: human beta2 microglobulin; -RT: RT-PCR without conversion of RNA into cDNA.

FIG. 2: Oct4 in co-cultured P19 EC and human Mesenchymal Stem Cells. Lane M: Mw markers; lanes hMSC: human Mesenchymal Stem Cells; lanes P19: P19 EC cells; lanes CO: P19 EC and hMSC co-cultured for 5 days; lane beta-2 and GAPDH: expression of human-specific beta-2 microglobulin and mouse GAPDH expression in co-cultured cells; lane beta-2 and GAPDH-RT: PCR without conversion of RNA in cDNA. HuOCT, hUCT4 28cy and 32 cy: human OCT4 expression and human OCT4 expression after 28 and 32 PCR cycles; h/m Oct4: expression of mouse and human Oct4.

FIG. 3: SSEA4 labeling of human Mesenchymal Stem Cells and human Mesenchymal Stem Cells in co-culture with P19 EC cells. SSEA4: SSEA4 staining. DiI: human hMSC labeled with DiI in co-culture with P19 EC cells. DAPI: visualization of nuclei. Transmission: visualization of cells. The SSEA4, DAPI and transmission pictures represent the same microscopic field and can be superimposed.

FIG. 4: In vitro translated UTF1 binds to the sequence of the SMAD binding element (SBE). In vitro translated Myc-UTF1 (indicated as myc cl 8.8) was directly Western blotted (1% of total) or Western blotted after binding to biotinylated SBE followed by precipitation using avidin-conjugated agarose beads. Myc-UTF1 (myc-clone 8.8) was detected by anti-Myc antibody in the total lysate as well as after binding to the SBE oligonucleotide sequence.

FIG. 5: UTF1 blocks TGF-beta and SMAD3/4-dependent transactivation of the (SBE)4-LUC reporter. SBE: transfection of SBE alone; TGF; SBE transactivation following TGFbeta stimulation; TGF-beta C18.8: TGF-beta induced SBE transactivation in the presence of over-expressed UTF1. Lanes S3/4: SBE transactivation following over-expression of Smad3/4, Smad3/4 and stimulation by TGF-beta, over-expression of Smad 3/4 in the presence of over-expressed UTF1 (cl 8.8) and over-expressed Smad3/4, over-expressed of UTF1 and stimulation with TGF-beta. SBE transactivation is indicated as fold induction over non SBE-LUC reporter transfected control cells.

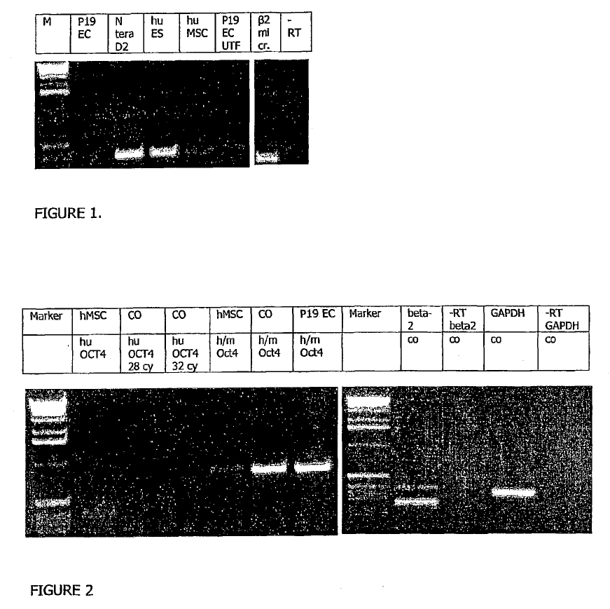

### DETAILED DESCRIPTION OF THE INVENTION

Further Experimental Procedures

Cell Culture and Media

(a) human Mesenchymal Stem Cells (for example as identified in U.S. Pat. No. 5,486,359). Human Mesenchymal Stem Cells were obtained from Poietics (BioWhittaker). Cells were grown in mesenchymal stem cell basal medium (MSCBM) supplemented with mesenchymal stem cell growth supplement, L-glutamine, Streptomycin and Penicillin according to the instructions of the supplier. Cells were cultivated for more then 15 passages without notable morphological alteration or change in marker expression. Cultures were maintained in 5% CO₂ at 37° C. is a humidified atmosphere.

(b) human Neuronal Progenitor Cells. Human Neural Progenitor Cells were obtained from Poietics (BioWhittaker). Cells were propagated as neurospheres in growth medium consisting of Neural Progenitor Maintenance Medium and recommended supplements (h-bFGF, h-EGF, neuronal survival factor, gentamicin and amphoceritin-B according to the instructions of the supplier. Cultures were maintained in 5% CO₂ at 37° C. is a humidified atmosphere.

(c) P19 and NteraD2 EC cells. P19 and NTeraD2 were obtained from the American Type and Culture Collection (ATCC) and cultured in alpha-minimal essential medium (alpha-MEM) supplemented with 7.5% Normal Calf Serum (NCS) and 2.5% Fetal Calf Serum (FCS). Medium was supplemented with penicillin (100 U/ml) and streptomycin (100 microgram/ml) and maintained in a 5% CO₂ atmosphere at 37° C.

(d) co-cultures of P19 EC and hMSCs. Cells were grown for 5 days in MSCBM (hMSCs and co-cultures) or alpha-MEM (P19 cells) on glass coverslips in 6-well plates. P19 cells monoculture: 5000 cell/well; hMSC monoculture 20,000 cells per well; co-culture: 3,000 P19 cells and 20,000 hMSCs per well.

Before plating cells for co-culture, a number of hMSCs were labeled using the life stain DiI (1,1″-dioctadecyl 3,3,3′,3′-tetramethylindocarbocyanine). Cells were labelled with DiI (5 μl/ml) for 10 minutes and washed 3 times with fresh medium. After staining the cells were kept in the dark to prevent decay of fluorescence.

2. Protocol for Charcoal Stripped Serum to Remove Retinoids

Day 1: Prepare dextran-coated charcoal suspension. Add 450 ml of tissue quality H₂O to 50 ml of TRIS/HCL 0.1M, pH 8.0. Dissolve in this buffer 0.25 gram of Dextran T500 Pharmacia, nr. 17.0320.01. Add 2.5 grams of activated charcoal Fluka cat.nr. 05120 or Sigma C-5260. Stir overnight at 4° C. in tightly locked vessel.

Day 2: Heat 200 ml of Fetal Calf Serum (FCS) for 30 minutes at 56° C. in a water bath. In the meantime fill 12×50 ml (plastic) centrifuge tubes with the charcoal suspension. Spin in swing-out rotor, 20 min, 1000×g. Discard the supernatant. Remove as much fluid as possible without touching the pellets. Add the serum to 6 charcoal pellets and resuspend the pellets in the serum. Incubate in a water bath at 45° for 45 mm, while shaking. Spin in swing-out rotor, 20 min, 1000×g. Add the serum supernatant to the 6 remaining charcoal pellets and repeat the whole procedure. Add the serum to 6 clean centrifuge tubes and spin again. Pool the serum in a clean bottle and filter/sterilize before freezing.

Retinoids or retinol derivatives are for example all-trans-retinyl esters, all-trans-retinol, 3,4-didehydro-retinol, 4-oxo-retinol, all-trans-retinal, 4-oxo-retinal, beta-carotene, all-trans-retinoic acid, 18-hydroxy-retinoic acid, 4-hydroxy-retinoic acid, 4-oxo-retinoic acid, 9-cis-retinoic acid, or 9-cis-4-oxo-retinoic acid.

3. Antibodies and Immunofluorescence.

Cells grown on cover slips were fixed in 4% paraformaldehyde in PBS for 30 min., washed with PBS and incubated for 1 hour at room temperature with the primary antibody. The primary antibodies SSEA1, SSEA4 and OCT4 were used at dilutions of 1:50; 1:50 and 1:100, respectively. The source of the antibodies were for SSEA1 (MC-480) and SSEA4 (MC-813-70) the Developmental Studies Hybridoma Bank, Iowa (USA) and anti-Oct4 (SC9081; H-134) was obtained from Santa Cruz Biotechnology, Inc. (Santa Cruz, USA). After washing with PBS, antibody localization was performed by using rabbit anti-mouse (SSEA1/4) and goat anti-rabbit (OCT4) immunoglobulins conjugated to fluorescein isothiocyanate (Oregon Green®). Samples were analyzed on a Zeiss fluorescence microscope equipped with epifluorescence and a camera to record data.

4. Gene expression studies

(a) RNA isolation and eDNA synthesis. At the time of harvest, medium was aspirated and cells were lyzed using Ultraspec™ (Biotecx) or Trizol® (Gibco BRL). RNA was isolated according to the instructions of the suppliers. RNA concentration was determined by measuring OD260 and equal amounts of RNA of the indicated cell lines were subjected to conversion into cDNA. RNA was reversed transcribed using random primers and 15 units M-MuLV (Promega) in a reaction mixture containing 4 μl 25 mM MgCl₂, 2 μl of each of the four dNTPs at 10 mM and 0.5 μl RNasin (Promega) in a total volume of 20 μl. RNA and 0.5 μg random hexamers were pre-incubated for 10 minutes at 55-60°. Mixtures were incubated at 37° C. for 1 hour.

(b) PCR reactions. PCR was were performed using 2 μl cDNA, 2.5 μl 10× SuperTaq buffer (without magnesium), 0.25 μl forward primer, 0.25 μl reverse primer, 0.50 μl 10 mM dNTPs, 1.25 Units of SuperTaq polymerase. In the case of β2-microglobulin 1.5 mM MgCl₂ was added to the reaction mixture. For mouse UTF1 PCR, normal Taq polymerase (Roche) was used with a buffer containing MgCl₂. The conditions for the PCR reaction were: 5 minutes 94° C.; 28 cycles of 30 seconds 94° C., 30 seconds 60° C. and 30 seconds 72° C.; 10 minutes 72° C., using a PTC-200, Peltier Thermal cycler. PCR-fragments were run on a 2% agarose gel and visualized by ethidium bromide staining.

(c) PCR primers

| | |
|---|---|
| (1) | human OCT4: |
| | forward: CTCCTGGAGGGCGAGGAATC; |
| | reverse: CCACATCGGCCTGTGTATAT |
| (2) | mouse/human Oct4: |
| | forward: GAGTTGGTTCCACCTTCTCC; |
| | reverse: GACACCTGGCTTCAGACTTC |
| (3) | mouse UTF1: |
| | forward: GTAAGAGGAGGAGAGCTGOO; |
| | reverse: CAGACTCTGCCTACTTACC |
| (4) | β2-microglobulin: |
| | forward: CCAGCAGAGAATGGAAAGTC; |
| | reverse: GATGCTGCTTACATGTCTCG |
| (5) | mouse GAPDH: |
| | forward: ATCACCATCTTCCAGGAG; |
| | reverse: GGCATCCACAGTCCT |
| (6) | gp80: |
| | forward: CCAACCACGAAGGCTGTGCT; |
| | reverse: GCTCCACTGGCCAAGGTCAA |
| (7) | LIF-R |
| | forward: CAACCAACAACATGCGAGTG; |
| | reverse: GGTATTGCCGATCTGTCCTG |
| (8) | SOCS1: |
| | forward: ACGCACTTCCGOAGATTCC; |
| | reverse: TCCAGCAGCTCGAAGAGGCA |
| (9) | gp130 |
| | forward: CcACATACGAAGACAGACCA; |
| | reverse GCGTTCTCTGACAACACACA |

5. Isolation and Injection of Mouse Blastocysts.

Pre-implantation blastocysts were removed from the uteri of pregnant C57BL/6 mice on the third day of pregnancy according to established procedures. Human mesenchymal cells cultured according the instructions supplier (Poietics) were trypsinized in trypsin/EDTA (Poietics) and taken up in 1 ml BMSCM medium containing 10% charcoal treated fetal calf serum (FCS). Human bone marrow stem cells were quickly thawed from liquid nitrogen and resuspended in 10 ml of DMEM medium containing 10% charcoal-treated FCS and centrifuged for 2 minutes at 850 g. The pelleted cells were resuspended in 1 ml of DMEM supplemented with 10% charcoal-treated FCS. Approximately 20 cells were taken up by suction into a siliconied glass capillary with a diameter that allowed the cells to pass without damage. Approximately 10-12 cells were injected into the blastocoele of the blastocysts with the use of a Narashige micro-injector. The injected blastocysts were transferred into 200 μl DMEM/10% FCS onto a non-tissue culture grade dish. To prevent liquid evaporation, the incubation medium was covered by freshly distilled paraffin oil. Embryos were cultured overnight at 37° C. under a 5% CO₂ atmosphere in a humidified incubator. On the morning of the following day, the still non-adherent blastocysts were lysed in 100 microliter Ultraspec solution (BioTecx) for the isolation m of RNA. For immunofluorescence, the blastocysts were transferred with a glass capillary into 200 μl DMEM supplemented with 10% FCS on a compartmentalized tissue culture plastic dish, and cultured for another 24 hours in DMEM/10% FCS. The now adherent blastocysts were fixed for 10 minutes at room temperature with a freshly prepared 4% formaldehyde solution in PBS. After three washes, the fixed samples were covered by a solution containing 50 mMTris pH 7.4; 150 mM NaCl; 5 mM EDTA; 0.05% NP-40; 0.25% gelatin. For immunofluorescence, the cells were incubated with the first antibodies, washed and incubated with the secondary antibody. Immunofluorescence was recorded using an inverted Zeiss microscope equipped with epifluorescence illumination and a camera to record and store the data.

Western Blotting

Proteins were separated by SDS-PAA gelelectrophoresis and transferred to a nitrocellulose membrane (Bio-rad) by electroblotting. The membrane was blocked for 30 min. in PBS containing 10% milk (Campina). After incubation with the primary antibody anti UTF1 1:200; anti-myc 1:500) for 1-2 hours, the membrane was washed 3 times with TBST. Species specific antibodies (1:3000) were incubated for 1 hour followed by washing with TBSB. The membrane was then incubated with a chemiluminescentie (ECL) solution (Roche) and revealed by the manufacturer's solution.

Transient and Stable Transfection

Cells were transfected with 10 microgram of plasmid DNA in 6-well plates using the Ca-phosphate co-precipitation method. After incubation with precipitate for 24 hours, the cells were washed with PBS and the medium was changed for new medium. Cells were lysed with 200 microliter of lysis buffer (Promega). Luciferase assays were performed according instructions of the supplier (Promega). Each transfection was carried out in triplicate.

To isolate stable transfectants, the pSV2Neo vector containing the neomycin resistance gene was transfected into P19 cells by calciumphosphate precipitation together with a plasmid containing the gene of of interest. Stable tranformants were selected with 400 micrograms of neomycin (G418) per ml. Colonies were picked with colony-rings.

### EXAMPLES

Dedifferentiation Experiments

Mouse

Hematopoietic stem cells isolated from embryonic liver of B6.5JL-Ly5.1 and eGFP transgenic mice are purified by Fluorescent Activated Cell Sorting (FACS). Hematopoietic stem cells are collected on the basis of Ly5.1 expression. Pools of approximately 100 cells are injected in blastocysts of congenic C57BL/6 strain of mice expressing the Ly5.2 allele. Injected blastocysts are disaggregated and Ly5.1 expressing cells are isolated by FACS and collected as single cells. Sorted cells are collected and maintained in media containing LIF and/or the MEK inhibitor. Oct4 expression is determined by single cell RT-PCR using murine Oct4-specific oligonucleotide primers.

Alternatively, mouse liver hematopoietic stem cells are mixed with microsurgically dissected inner cell mass cells of blastocysts followed by the procedures described above.

Human

Hematopoietic stem cells (CD34 positive) are injected into C57BL/6 or immunodeficient NOD-SCID or Rag−/− mouse blastocysts and expression of human Oct4 is determined by RT-PCR.

Blastocysts injected with CD34-positive human hematopoietic cells transduced with Oct4 promoter-eGFP fusion genes are assayed for eGFP and isolated by FACS.

FACS sorted cells are maintained in LIF and MEK inhibitor containing media.

1. Dedifferentiation of Human Somatic Stem Cells Following Micro-Injection in Mouse Blastocysts

To investigate whether the inner cell mass of mouse pre-implantation embryos constitutes an environment which induces dedifferentiation of somatic stem cells to ES-equivalent cells, two human stem cell types i.e., human Mesenchymal Stem Cells (hMSC) and human Hematopoietic Stem Cells (AC133+from cord blood, Poietics (BioWhittaker) were injected into mouse 3.5 day blastocysts. Between 10-12 cells were injected into the blastocoel. After culturing the injected blastocysts for 24 hours, RNA was isolated from the injected as well as control embryos and analyzed for the expression of human OCT4. The remainder of the injected blastocysts was cultured for an additional 24 hours and prepared for immunofluorescence.

Analysis of OCT4 Expression

(a) design of a Human-Specific OCT4 Primer Set

To discriminate between human and mouse Oct4 transcripts in mixed mouse/human RNA samples containing both Oct4 orthologs, a human-specific OCT4 primer set was designed. The two primers of the human-specific primer set are located on separate exons and results in amplification of a human of 380 bp with RNA from human cells but not with RNA mouse cells. The UTF-1 transcript detected in RNA of mouse P19 EC cells indicates that the mouse RNA is intact. In RNA samples from human cells, the -RT reactions generates a much larger fragment indicative for amplification from genomic DNA. This band is not present in the human cell derived RNA samples. Beta-2 microglobulin expression is used as internal control.

(b) Oct4 Expression in hMSC's and hHSC's Injected in Mouse Blastocysts

Using the human-specific OCT4 primer set, OCT4 expression was analyzed in RNA samples isolated from the injected blastocysts. As summarized in Table 1, the injected hMSC express of OCT4. When normalized against the expression of beta2 microglobulin in hMSC control the level of expression of OCT4 is increased in hMSC and hHCS injected into blastocysts. By using the m/h OCT4 primer set, Oct4 expression is present in mouse, human and mixed RNA samples confirming the integrity of the RNA samples.

(b) SSEA4 Expression by Blastocyst-Injected hMSC and hHSC

Unlike the cells of the inner cell mass of the mouse blastocysts, both hMSC and hHSC show immunoreactivity against SSEA4 (Table 2). These results indicate that the blastocyst injected adult stein cells exhibit properties of ES-like cells as a result of incubation within the environment of the pre-implantation embryo.

### Example 2

Dedifferentiation of Human Mesenchymal Stem Cells by Co-Culture With P19 EC Cells

The inner cell mass of pre-implantation mouse blastocysts represents an environment that leads to dedifferentiation of adult or somatic stem cells representative of an ES cell-like state. Cells derived or resembling the inner cell mass of mouse blastocysts like undifferentiated EC cells may exhibit a similar property. This property of EC cells can be demonstrated by co-culture of undifferentiated mouse P19 EC and hMSCs. P19 EC and hMSC were plated at different initial cell densities to accommodate for the different growth rate of both cell types. After 5 days of co-culture, the cells were analyzed for the expression human ES cell-specific markers by immunofluorescence and RT-PCR.

(a) analysis of SSEA4 Expression

Human mysenchymal stem cells in co-culture with P19 EC cells express the human-specific EC/ES cell marker SSEA4, while this marker is hardly detectable in hMSCs in monoculture. To discriminate between P19 and hMSC in co-culture, the hMSC were labeled with the life stain DiI. The co-localization of DiI and SSEA4 confirms that SSEA4 expression is resulting from the hMSCs in the co-cultured cells (FIG. 2). As control, the co-cultures were stained with SSEA1, which stains mouse P19 cells but not the hMSC. Results of this study are summarized in Table 3.

(b) OCT4 Expression in Co-Cultures of P19 and hMSC

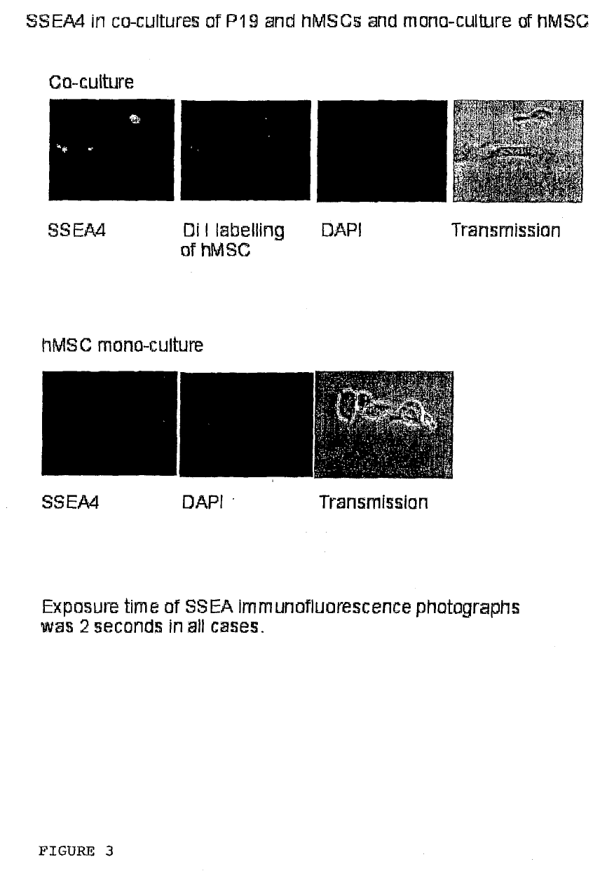

Expression of human OCT4 expression was analyzed in RNA of co-cultured P19 and hMSCs. As shown in FIG. 3, human OCT4 is expressed at low levels in hMSC but could not be detected in the co-cultured hMSC even after 28 and 32 PCR cycles. In contract, the mouse/human primer set shows expression of Oct4 in both human MSCs and mouse P19 cells as well as in the co-cultured cells.

These combined results from the immunofluorescent and gene expression studies indicate that in co-culture with P19 cells, a small percentage of hMSCs has dedifferentiated to an ES-like cell expressing the stem cell marker SSEA4. A larger fraction of hMSCs have differentiated into a cell type in which OCT4 is no longer expressed biasing the detection of human OCT4 transcripts in mixed mouse/human RNA samples derived from the co-cultured cells.

### Example 3

Inhibition of HDAC Expression by TSA

Histon de-acetyelase (HDAC) activity has been shown to repress gene transcription through de-acetylation of histons, keeping chromatin in a condensated state. HDAC activity is inhibited by Trichostatin A (TSA). Human mesenchymal stem cells and mouse and human neuronal progenitor cells were treated by TSA at concentration of 10 and 50 ng/ml. After 24 and 48 hours, OCT4 expression was analyzed by RT-PCR.

TSA treament for 24 to 48 hours enhances the expression of OCT4 in both human MSC and human Neural Progenitor Cells as well as in mouse neural progenitor cells. (Table 3). In line with the OCT4-UTF1-SMAD relationship, expression of UTF1 was also increased leading to a reduction in TGF-beta, activin and BMP responsiveness of the TSA treated cells. Furthermore SSEA4 expression was increased in parallel with upregulation of OCT4, indicating that upregulation of OCT4 resulted in dedifferentiation of both human stem cell types. TSA treated cells were allowed to form embroid bodies by plating the cells on non-tissue culture grade plastics or in hanging droplets in the presence of retinoic acid or 1% DMSO. Visual inspection of the re-plated cultures revealed cells of different morphology compared to the parental cultures indicative for the appearance of ectoderm and mesoderm differentiated derivatives.

### Example 4

Dedifferentiation of hMSC by Long Term Culture in Defined Media

Human MSC were grown on fibronectin coated dishes in DMEM containing PDGF-BB (0,1-100 microgram/ml), EGF (0.1 to 100 microgram/ml), dexamethason (10-7-10-8 mM), ascorbic acid (0.1 to 10 mM), linoleic acid (0.1 to 10 microgram/ml) supplemented with 2% charcoal treated Fetal Calf serum (FCS) and cultured at densities between 103 to 5×10³ cells per cm². At initial plating, hMSCs express low levels of the human ES cell markers Oct4 and SSEA4. Cells cultured in this medium for more then 25 population doublings exhibit gradually increased Oct4 expression as determined by semi-quantitative PCR as well as increased SSEA4 immunoreactivity (Table 4). Under these culturing conditions hMCS adopts a more dedifferentiated phenotype resembling that of human ES cells. The dedifferentiated (ES-equivalent or ES-like cells) can be differentiated in vitro into a variety of cell types including skeletal, smooth and cardiac muscle by treatment with 5-aza-cytidine, retinoic acid and BMP plus bFGF, respectively. In addition, endothelial cells, hematopoietic precursers and mature blood cells, osteoblasts, chondroblasts and neuronal cell types including neurons, astrocytes and glia can be derived from these cells using procedures that are commonly used in obtaining these differentiated derivatives from ES cells.

### Example 5

LIF Responsiveness and Expression Among Stem Cell Lines

Gp 80 is Differential Expressed.

Expression of genes that are part of the LIF signal transduction pathway, including LIF receptor, gp130, SOCS1, STAT3 and IL-6 receptor gp8O was investigated in human ES cells, the human EC cell line NteraD2 and h.MSC Iable.

In all three cell lines, LIF receptor, gp 130 and STAT3 are expressed at comparable levels (Table 5). In human NteraD2 cells LIF-induced STAT3 tyrosine 705 is blocked. The high level expression of SOCS1, which inhibits the JAK-2 tyrosine protein kinase may be responsible for the observed LIF resistance of human EC and ES cells. hMSCs express IL-6 receptor gp80. Since hES cells do not express gp8O, loss of expression of this gene is a marker for dedifferentiation of these into an ES-like cell.

### Example 6

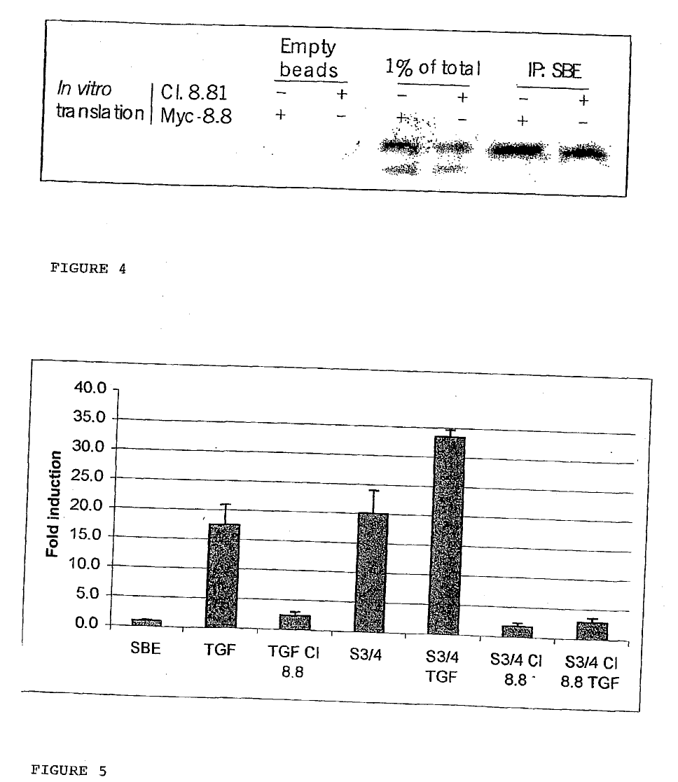

The transcription factor UTF1 binds to the sequence CAGACAG referred to as SMAD binding element (SBE) as identified in the JunB promoter (Jonk et al.). UTF1 (indicated as clone 8.8 in FIG. 4) was in vitro translated as a Myc-UTF1 fusion protein and allowed to form a DNA-protein complex with a double stranded biotinylated CAGAGACGTCTCTG probe and protein binding was detected by Western blotting.

Overexpression of UTF1 blocks Smad-dependent transactivation of the JunB (SBE)4-LUC reporter in transient transfection assays. Transient overexpression of UTF1 blocks Smad1 plus Smad4, Smad2 plus Smad4 and Smad3 plus Smad4 transactivation both in the absence as well as in the presence of a stimulary ligand (TGF-beta, Activin, BMP) (FIG. 5). UTF1 elicited repression of SMAD-dependent transactivation is observed in transiently transfected cells that endogenously express UTF1 (P19 EC, NteraD2) as well as in cells in which UTF1 is not expressed.

Stable expression of UTF1 in P19 EC cells or a clonally isolated variant of this cell line blocks RA- and DMSO-induced differentiation as indicated by maintenance of expression of SSEA1 immunoreactivity of the UTF1 expressing P19 cells up to several days after the induction of differentiation. Constitutive expression of UTF1 fails to induce the expression of Smad-regulated target genes by TGF-beta, BMP and Activin and related family members.

TABLE 1
Analysis of human Oct4 expression in human Mesenchymal Stem
Cells (hMSC) and human Hematopoietic Stem Cells (hHSC) injected in
3.5 day mouse blastocysts.
A

| | | P19 | | | | | |
|---|---|---|---|---|---|---|---|
| | Control | EC | hMSC | hHSC | hMSC | NTera | |
| | No Inj. | cells | Inj. | Inj. | No. inj. | D2 | −RT |
| human | − | − | + | + | +/− | ++ | − |
| OCT4 | | | | | | | |
| hu beta-2 | − | − | + | + | + | + | + |
| micro- | | | | | | | |
| globulin | | | | | | | |

B

| | | P19 | | | | | |
|---|---|---|---|---|---|---|---|
| | Control | EC | MSCs | MSC | hMSCs | NTera | |
| | No Inj. | cells | Inj. | Inj. | No. inj. | D2 | −RT |
| m/h Oct4 | + | + | + | + | + | + | − |
| Mgapdh | + | + | − | − | − | − | − |

(A) human OCT4 and beta 2 microglobulin expression in non-injected embryo (control, no inj.), P19 EC cells, injected hMSC, injected hHSC, not injected hMSC, and non injected NteraD2. Lane −RT is PCR on non-reverse transcriptase treated RNA.
(B) mouse and human Oct4 and mouse GAPDH expression in same samples as under (A).
(−) non expression;
(+/−) expression detectable;
(+) clear expression;
(++) strong expression.

TABLE 2
SSEA4 staining of hMSC and hHSC injected into 3.5 day mouse
blastocysts and non-injected cells.

| | Control | hMSC | hHSC | | |
|---|---|---|---|---|---|
| | blastocysts | injected | injected | hMSC | hHSC |
| SSEA4 | − | + | + | − | − |
| DAPI | nuclei | nuclei | Nuclei | nuclei | nuclei |

Control blastocyst were non-injected.
DAPI was used to stain nuclei.

TABLE 3
Analysis of SSEA4 expression in human Mesenchymal Stem Cells
(hMSC) co-cultured with mouse P19 EC cells.

| | P19 + | | | | |
|---|---|---|---|---|---|
| Marker | hMSCs | | hMSCs | Remarks | |
| SSEA4 | − | + | +/− | Expression of | Marks hMSCs |
| | | | | SSEA4 slightly | |
| | | | | increased | |
| SSEA4 (DiI) | − | ++ | +/− | Compared to | Marks hMSCs |
| | | | | mono-cuilture | specifically |
| | | | | SSEA4 | |
| | | | | expression had | |
| | | | | increased | |
| | | | | clearly | |
| SSEA1 | + | − | − | | Marks P19 |

Controls: SSEA1 expression in mouse P19 EC cells and SSEA4 expression in hMSC.
(−) no expression;
(+/−) expression detectable;
(+) clear expression;
(++) strong expression

TABLE 4
OCT4 expression after TSA treatment.

| | | | |
|---|---|---|---|
| | | | mNPC |
| TSA | hMSC | hNPC | (eGFP/bcl2) |

| Treatment | 10 | 50 | 10 | 50 | 10 | 50 |
|---|---|---|---|---|---|---|
| 0 | +/− | +/− | +/− | +/− | +/− | +/− |
| 24 hours | + | + | + | + | + | + |
| 48 hours | ++ | ++ | ++ | ND | ++ | ND |

hMSC: human Mesenchymal Stem Cells;
hNPC: human Neural Progenitor Cells;
mNPC: mouse Neural Progenitor Cells isolated from day 14 mouse brain of eGFP and bcl2 transgenic mice.
TSA was used at 10 and 50 ng/ml.
Oct4 expression was determined by RT-PCR and quantified as follows:
+/− expression detectable;
(+) clear expression;
(++ strong expression.
ND: not determined.

TABLE 5
Long-term culture of hMSC in dedifferentiation inducing media.

| hMSC | | | | BMP/TGF- | |
|---|---|---|---|---|---|
| population | Oct4 | SSEA4 | UTF1 | beta/Activin | LIF- |
| doubling | expression | staining | expression | response | response |
| 2 | very low | very low | very low | + | − |
| 10 | +/− | low | +/− | +/− | − |
| 30 | + | moderate | + | +/− | − |
| 50 | ++ | strong | ++ | − | − |

OCT4 expression was determined by RT-PCR.
TJTF1 expression was determined by Western blotting.
SSEA4 expression was determined by immunofluorescence.
BMP/TGF-beta/activin response was determined by transient transfection of the (SBE)4-luc reporter.
LF responsiveness was determined by analysis of STAT3 tyr 705 and STAT3 ser 727 phosphorylation using STAT3 phosphospecific antibodies.
(−) no expression or no response;
(+/−) low expression or response;
(+) clear expression or response;
(++) strong expression or response.

TABLE 6
Expression of genes involved in LIF signal transduction in human and
mouse ES and EC cells by RT-PCR.

| | Human ES | NTeraD2 | hMSC | P19 EC |
|---|---|---|---|---|
| m/h LIF | ++ | ++ | ++ | ++ |
| receptor | | | | |
| m/h gp80 | − | + | + | + |
| (IL-6 receptor) | | | | |
| m/h SOCS1 | +++ | +++ | +++ | − |
| m/h STAT3 | ++ | ++ | ++ | ++ |
| h b2 microgl. | ++ | ++ | ++ | − |

Human ES (human embryonic stem cells;
NteraD2: human Embryonal Carcinoma cells;
hMSC: human Mesenchymal Stem Cells;
P19 EC: mouse embryonal carcinoma cells.
m/h indicates that the primers hybridize with sequences of both human and mouse orthologs.
(−) no expression;
(+/−) expression detectable;
(+) clear expression;
(++) strong expression
gp80: glycoprotein 80 or IL-60 receptor;
SOCS1: suppressor of cytokine signaling 1;
STAT3: signal tranducer and activator of transcription3;
b2 micr: beta2-microglobulin.

What is claimed is:

1. A method for obtaining a stem cell-like cell from a sample taken from a multicellular organism, said method comprising:
culturing cells from said sample and allowing for transcription, translation or expression by at least one of said cells of a gene or fragment thereof that, in general, is differentially expressed at different phases of embryonic development, and obtaining a stem cell-like cell.

2. The method according to claim 1 wherein said organism is functionally differentiated.

3. The method according to claim 1 or claim 2 wherein said organism is a vertebrate.

4. The method according to any one of claims 1 to 3 further comprising culturing cells from said sample in the relative absence of a differentiation factor

5. The method according to claim 4 wherein said differentiation factor has retinoid activity.

6. The method according to any one of claims 1 to 5 wherein said gene is overexpressed in an early phase of embryonic development.

7. The method according to claim 6 wherein said early phase comprises the blastula stage.

8. The method according to claim 7 wherein, in mammals, said blastula stage comprises a pre-implantation stage.

9. The method according to any one of claims 1 to 8 wherein said gene comprises Oct4 or orthologue thereof.

10. The method according to any one of claims 1-9 further comprising:
selecting said stem cell-like cell by detecting expression of cell surface markers stage specific embryonic antigen.

11. The method according to claim 10 wherein said cell surface markers stage specific embryonic antigen comprises SSEA-1, SSEA-3, SSEA-4, TRA-1-60, TRA-1-81 and/or alkaline phosphatase or analogue thereof.

12. A cell wherein said cell is a dedifferentiated stem cell.

13. A cell wherein said cell is a stem cell-like cell produced by a method according to any one of claims 1-11.

14. The cell of claim 12 or claim 13 comprising a recombinant nucleic acid.

15. A culture comprising the cell of claim 12, claim 13, or claim 14.

16. A graft or transplantation material comprising the cell of claim 12, claim 13, or claim 14 or the culture of claim 15.

17. A non-human animal comprising the cell of claim 12, claim 13 or claim 14 or the culture of claim 15.

18. A pharmaceutical composition for treating a subject with a graft, said pharmaceutical composition comprising the cell of claim 12, claim 13, or claim 14 or the culture of claim 15.

19. The pharmaceutical composition of claim 18 wherein the subject or a sample taken therefrom comprises a source of the graft.

20. A method of cloning a non-human animal, the improvement comprising using in said method the cell of claim 12, claim 13, or claim 14 or the culture of claim 15 in the cloning of the non-human animal.

21. The according to claim 20 wherein said non-human animal is an experimental animal, a farm animal, or an animal for xenotransplant production.
